(19) United States
(12) Patent Application Publication
(10) Pub. No.: US 2013/0164746 A1
(43) Pub. Date: Jun. 27, 2013

(54) Mutations in SF3B1 and Chronic Lymphocytic Leukemia

(71) Applicants: Davide Rossi, D'agogna (IT); Gianluca Giadano, Novara (IT); Robert Foa, Rome (IT)

(72) Inventors: Davide Rossi, D'agogna (IT); Gianluca Giadano, Novara (IT); Robert Foa, Rome (IT)

(21) Appl. No.: 13/631,972

(22) Filed: Sep. 29, 2012

Related U.S. Application Data
(60) Provisional application No. 61/540,618, filed on Sep. 29, 2011.

(51) Int. Cl. C12Q 1/68

(52) U.S. Cl. CPC C12Q 1/6886
USPC 435/6.11

(57) ABSTRACT

The disclosure provides methods of prognosing a subject with CLL and determining the response of the subject to treatment with fludarabine by determining the presence or absence of mutations within the SF3B1 gene.

### RELATED APPLICATIONS

This application claims benefit of priority from U.S. Provisional Patent Application 61/540,618, filed Sep. 29, 2011, which is hereby incorporated in its entirety as if fully set forth.

### FIELD OF THE DISCLOSURE

The present disclosure relates generally to the fields of molecular biology, genetics, and cancer. Specifically, mutations in the SF3B1 gene are used to diagnose, prognose, and determine optimal treatment regimens for subjects with chronic lymphocytic leukemia.

### BACKGROUND OF THE DISCLOSURE

Chronic lymphocytic leukemia (CLL) is cancer of white blood cells called lymphocytes. As CLL progresses, the number of B lymphocytes or B cells present in the bone marrow increases. These cancerous B cells migrate or spread from the bone marrow into the blood. Via the blood these cancerous cells have access to all organs in the body. Most commonly, cancerous B cells affect lymph nodes, liver, and spleen. Ultimately the bone marrow fails to function properly, leading to death.

The clinical course of chronic lymphocytic leukemia (CLL) ranges from a very indolent disorder with a normal lifespan, to a rapidly progressive disease that ultimately becomes chemorefractory and leads to death. Occasionally, CLL undergoes histological transformation to Richter syndrome (RS).

The variable clinical course of CLL is driven, at least in part, by the molecular heterogeneity of the disease. Despite recent advances, the genetic lesions identified to date do not fully recapitulate CLL molecular pathogenesis and do not entirely explain the development of severe complications, such as chemorefractoriness and RS transformation, which still represent an unmet clinical need.

Identification of genetic lesions associated with chemorefractoriness represents a critical step for the early identification of high risk CLL patients and for the development molecularly tailored drugs.

### SUMMARY OF THE DISCLOSURE

The compositions and methods of the disclosure provide a solution to the long-felt and unsolved need for a biological indicator of disease progression and responsiveness to treatment. The disclosure provides missense and deletion mutations within the SF3B1 (splicing factor 3b, subunit 1, 155 kDa) gene that change the amino acid sequence of the encoded protein. These changes in the protein have functional consequences. SF3B1 encodes subunit 1 of the splicing factor 3b protein complex. Under normal or wild-type conditions, splicing factor 3b, together with splicing factor 3a and a 12S RNA unit, forms the U2 small nuclear ribonucleoproteins complex (U2 snRNP). The splicing factor 3b/3a complex binds pre-mRNA. Splicing factor 3b is also a component of the minor U12-type spliceosome. Thus, subunit 1 of the splicing factor 3b protein complex plays a number of critical roles in the splicing mechanism of the cell. Mutations in SF3B1 affect the ability of a cell to convert pre-mRNA, which contains intronic sequence, into mature mRNA. In the context of CLL, these mutations are predictive of decreased survival in patients and increased resistance to treatment with fludarabine

The disclosure provides a method of prognosing a subject with chronic lymphocytic leukemia (CLL), comprising, (a) obtaining a biological sample from the subject; (b) determining the sequence of a portion of the SF3B1 gene in the sample, wherein the portion of the SF3B1 gene comprises a sequence that encodes for the HEAT3, HEAT4 or HEAT5 domain; (c) analyzing the sequence of the SF3B1 gene for a mutation, wherein the presence of the mutation in the sequence of the SF3B1 gene predicts a decreased survival of the subject, thereby prognosing the subject with chronic lymphocytic leukemia (CLL). In many embodiments, the mutation is present within the HEAT3, HEAT4 or HEAT5 domain. In further embodiments, the method comprises preparation of a nucleic acid molecule from a subject followed by analysis as disclosed herein to detect nucleic acid alterations that predict or forecast the probable course and/or outcome of CLL.

Alternatively, or in addition, the disclosure provides a method of prognosing a subject with chronic lymphocytic leukemia (CLL), comprising, (a) obtaining a biological sample from the subject; (b) determining the sequence of a portion of the SF3B1 polypeptide in the sample, wherein the portion of the SF3B1 polypeptide comprises the sequence of a HEAT3, HEAT4 or HEAT5 domain; (c) analyzing the sequence of the SF3B1 polypeptide for a mutation, wherein the presence of the mutation in the sequence of the SF3B1 polypeptide predicts a decreased survival of the subject, thereby prognosing the subject with chronic lymphocytic leukemia (CLL). In many embodiments, the mutation is present within the HEAT3, HEAT4 or HEAT5 domain. In further embodiments, the method comprises preparation of a polypeptide molecule from a subject followed by analysis as disclosed herein to detect amino acid alterations that predict or forecast the probable course and/or outcome of CLL.

With respect to methods of prognosing a subject, the term decreased survival includes treatment-free survival or overall survival. Embodiments of the invention include predicting or forecasting the probable course and/or outcome of CLL in a subject with the nucleic acid, or amino acid, alteration in the absence of treatment for CLL. In other embodiments, the probable course and/or outcome is for a subject with the nucleic acid, or amino acid, alteration if treated with a disclosed treatment for CLL.

The disclosure also provides a method of determining the response of a subject with chronic lymphocytic leukemia (CLL) to treatment with fludarabine, comprising, (a) obtaining a biological sample from the subject; (b) determining the sequence of a portion of the SF3B1 gene in the sample, wherein the portion of the SF3B1 gene comprises a sequence that encodes for a HEAT3, HEAT4 or HEAT5 domain; (c) analyzing the sequence of the SF3B1 gene for a mutation, wherein the presence of the mutation in the sequence of the SF3B1 gene indicates that the subject is resistant or refractory to fludarabine, thereby determining the response of the subject with chronic lymphocytic leukemia (CLL) to treatment with fludarabine. In many embodiments, the mutation is present within the HEAT3, HEAT4 or HEAT5 domain. In further embodiments, the method comprises preparation of a nucleic acid molecule from a subject followed by analysis as disclosed herein to detect nucleic acid alterations that predict or forecast the probable non-responsiveness of CLL in the subject to treatment with fludarabine.

Alternatively, or in addition, the disclosure provides a method of determining the response of a subject with chronic lymphocytic leukemia (CLL) to treatment with fludarabine, comprising, (a) obtaining a biological sample from the subject; (b) determining the sequence of a portion of the SF3B1 polypeptide in the sample, wherein the portion of the SF3B1 polypeptide comprises the sequence of a HEAT3, HEAT4 or HEAT5 domain; (c) analyzing the sequence of the SF3B1 polypeptide for a mutation, wherein the presence of the mutation in the sequence of the SF3B1 polypeptide indicates that the subject is resistant or refractory to fludarabine, thereby determining the response of the subject with chronic lymphocytic leukemia (CLL) to treatment with fludarabine. In many embodiments, the mutation is present within the HEAT3, HEAT4 or HEAT5 domain. In further embodiments, the method comprises preparation of a polypeptide molecule from a subject followed by analysis as disclosed herein to detect amino acid alterations that predict or forecast the probable non-responsiveness of CLL in the subject to treatment with fludarabine.

In certain aspects of this method, the portion of the SF3B1 polypeptide comprising the sequence of a HEAT3, HEAT4 or HEAT5 domain is selected from the group consisting of SEQ ID NO: 1, 2, 3, 4, 5, 6, 7, 8, 9, 10, 11, 12, 13, 14, 15, 16, and 19. In some embodiments, the portion of the SF3B1 polypeptide comprising the sequence of a HEAT3, HEAT4 or HEAT5 domain is SEQ ID NO: 1 or 19.

With respect to methods of determining the response of a subject with chronic lymphocytic leukemia (CLL) to treatment with fludarabine, in subjects who carry one or more mutations in either the SF2B1 gene or polypeptide, the treatment with fludarabine is discontinued or replaced by treatment with chlorambucil, cyclophosphamide, rituximab, alemtuzumab, bendamustine, or a combination thereof. In many embodiments, the treatment with fludarabine is replaced by treatment with alemtuzumab. In other embodiments, treatment with fludarabine is not initiated, and instead, the subject is treated with chlorambucil, cyclophosphamide, rituximab, alemtuzumab, bendamustine, or a combination thereof. Therefore, the disclosure further includes a method of treating a subject with CLL comprising determination of responsiveness to fludarabine treatment as disclosed herein and discontinuing or altering the treatment as described above. In additional embodiments, the disclosure includes a method of treating a subject with CLL comprising determination of responsiveness to fludarabine treatment as disclosed herein and initiating treatment for the CLL with a therapy other than fludarabine as described above.

With respect to any method of the disclosure, the analyzing step may include polymerase chain reaction (PCR), Sanger sequencing, next generation sequencing, or a combination thereof as known to the skilled person. In some embodiments, the analysis may use prepared or isolated DNA molecules that are used as templates or for hybridization. In some cases, a DNA molecule as template is amplified, such as by PCR or quantitative PCR, with optional detectable labeling of the amplified molecules to aid in their detection. In other cases, the amplified molecules may be detected based upon hybridization to a polynucleotide probe. In alternative cases, a prepared or isolated DNA molecule is not copied or amplified, but directly sequenced instead, with optional direct or indirect immobilization on a solid support or solid phase medium, prior to sequencing. Non-limiting examples of a solid support or solid phase medium include a bead, a microbead, or other insoluble material. In further cases, a DNA molecule may be prepared or isolated by incorporation into, or as part of, an emulsion or a compartment, such as a droplet or microdroplet, or other suspension in solution.

In the practice of the disclosure, the subject may be diagnosed with chronic lymphocytic leukemia (CLL) or may not be diagnosed with chronic lymphocytic leukemia (CLL). These methods can be applied at any point in the diagnosis or treatment of a subject. Subjects with CLL may present one or more of the following non-limiting list of symptoms: enlarged lymph nodes, liver, or spleen; excessive sweating or night sweats; fatigue; fever; recurring infections; and unintentional weight loss. Subjects with CLL may present a higher-than-normal white blood cell count, anemia and/or thrombocytopenia. Subject may be treated with one or more of the following non-limiting exemplary treatments: fludarabine (Fludara), chlorambucil, cyclophosphamide (Cytoxan), rituximab (Rituxan), alemtuzumab (Campath), bendamustine, or a combination thereof. Subjects who are resistant or refractory to treatment with fludarabine because they carry one or more of the mutations described herein may be treated with Alemtuzumab (Campath). Subjects who carry one or more of the mutations described herein may be also treated with Bendamustine, particularly when the CLL returns after an initial treatment or when the subject or patient suffers a relapse.

In the practice of the disclosure, the biological sample includes an isolated and purified genomic DNA, cDNA, or RNA molecule. Alternatively, the biological sample includes an isolated and purified polypeptide molecule. The biological sample can be obtained from one or more tissues or bodily fluids. For mutation detection, exemplary tissues include, but are not limited to, bone marrow, blood cells, peripheral blood cells, lymph nodes, spleen, muscle tissue (including, smooth, visceral, striated, skeletal, or cardiac muscles composed of muscle cells or fibers), nervous system tissue (including, but not limited to, the neurons and glia or the central and peripheral nervous system), or epithelial tissues (including, but not limited to, epithelial cells that comprise the skin, respiratory tract, reproductive tract, and digestive tract). To confirm the somatic origin of the mutations, exemplary specimens include saliva, epidermal cells obtained by a non-invasive scraping of the skin or a swab of the inner cheek. In some embodiments, the tissue sample comprises red or white blood cells isolated from whole blood. Exemplary bodily fluids include, but are not limited to, aqueous humour, vitreous humour, bile, whole blood, blood serum, breast milk, cerebrospinal fluid (CSF), endolymph, perilymph, gastric juice, mucus (including nasal drainage and phlegm), peritoneal fluid, pleural fluid, saliva, sebum (skin oil), sweat, tears, and urine. In some embodiments, the bodily fluid is whole blood, blood serum, endolymph, perilymph, saliva, or urine.

In the practice of the disclosure, the SF3B1 mutation may be a missense mutation or an in-frame deletion in the polynucleotide or polypeptide sequence in a SF3B1 gene or the resultant polypeptide encoded by a SF3B1 gene, respectively. In many embodiments, a missense mutation or an in-frame deletion is in a HEAT3, HEAT4, or HEAT5 domain.

In some disclosed methods of prognosing a subject, the mutation may be a substitution of a Guanine (G) for an Adenine (A) at nucleotide base position 2044 or 2146; a substitution of a Thymine (T) for a Guanine (G) at nucleotide base position 2046; or a substitution of an Alanine (A) for a Guanine (G) at nucleotide base position 2267, of SEQ ID NO: 17 (Genbank Accession No. NM_{—}012433.2).

In other disclosed methods of prognosing a subject, the mutation may be a substitution of a Glutamic Acid (Glu or E) residue for a Lysine (Lys or K) residue at codon 666 or 700; results in a substitution of an Asparagine (Asn or N) residue for a Lysine (Lys or K) residue at codon 666; or a substitution of a Glutamic Acid (Glu or E) residue for a Glycine (Gly or G) residue at codon 740, of SEQ ID NO: 19 (Genbank Accession No. NP_{—}036565.2).

In some disclosed methods of determining the response of a subject to treatment with fludarabine, the mutation may be a substitution of a Thymine (T) for an Adenine (A) at nucleotide base position 1938; a substitution of an Adenine (A) for a Cytosine (C) at nucleotide base position 2034; a substitution of a Guanine (G) for a Cytosine (C) at nucleotide base position 2032; a substitution of a Guanine (G) for an Adenine (A) at nucleotide base position 2044; a substitution of a Guanine (G) for an Adenine (A) at nucleotide base position 2146; a deletion of the nucleotide sequence CAGAAA corresponding to nucleotide base positions 2143 to 2148; or a substitution of a Guanine (G) for a Cytosine (C) at nucleotide base position 2056, of SEQ ID NO: 17.

In some disclosed methods of determining the response of a subject to treatment with fludarabine, the mutation may be a substitution of a Serine (Ser or S) residue for an Arginine (Arg or R) residue at codon 630; a substitution of a Glutamine (Gln or Q) residue for a Histidine (His or H) residue at codon 662; a substitution of an Aspartic Acid (Asp or D) residue for a Histidine (His or H) residue at codon 662; a substitution of a Glutamic Acid (Glu or E) residue for a Lysine (Lys or K) residue at codon 666 or 700; a deletion of a Glutamine (Gln or Q) residue at codon 699 and a Lysine (Lys or K) residue at codon 700; a substitution of a Glutamic Acid (Glu or E) residue for a Glutamine (Gln or Q) residue at codon 670, of SEQ ID NO: 19.

In many disclosed methods of prognosing a subject, the mutation results in a substitution of a Glutamic Acid (Glu or E) residue for a Lysine (Lys or K) residue at codon 666 or 700. When either the K666E or K700E substitution is present, the mutation is a substitution of a Guanine (G) for an Adenine (A) at nucleotide base position 2044 or 2146, respectively. With respect to some disclosed methods of prognosing a subject, the mutation results in a substitution of an Asparagine (Asn or N) residue for a Lysine (Lys or K) residue at codon 666. When the K666N substitution is present, the mutation is a substitution of a Thymine (T) for a Guanine (G) at nucleotide base position 2046. With respect to other disclosed methods of prognosing a subject, the mutation results in a substitution of a Glutamic Acid (Glu or E) residue for a Glycine (Gly or G) residue at codon 740. When the G740E substitution is present, the mutation is a substitution of an Adenine (A) for a Guanine (G) at nucleotide base position 2267.

In other disclosed methods of determining the response of a subject to treatment with fludarabine, the mutation results in a substitution of a Serine (Ser or S) residue for an Arginine (Arg or R) residue at codon 630. When the R630S substitution is present, the mutation is a substitution of a Thymine (T) for an Adenine (A) at nucleotide base position 1938. In some disclosed methods of determining the response of a subject to treatment with fludarabine, the mutation results in a substitution of a Glutamine (Gln or Q) residue for a Histidine (His or H) residue at codon 662. When the H662Q substitution is present, the mutation is a substitution of an Adenine (A) for a Cytosine (C) at nucleotide base position 2034. In further disclosed methods of determining the response of a subject to treatment with fludarabine, the mutation results in a substitution of an Aspartic Acid (Asp or D) residue for a Histidine (His or H) residue at codon 662. When the H662D substitution is present, the mutation is a substitution of a Guanine (G) for a Cytosine (C) at nucleotide base position 2032. In additional disclosed methods of determining the response of a subject to treatment with fludarabine, the mutation results in a substitution of a Glutamic Acid (Glu or E) residue for a Lysine (Lys or K) residue at codon 666 or 700. When the K666E substitution is present, the mutation is a substitution of a Guanine (G) for an Adenine (A) at nucleotide base position 2044. When the K700E substitution is present, the mutation is a substitution of a Guanine (G) for an Adenine (A) at nucleotide base position 2146. In cases of disclosed methods of determining the response of a subject to treatment with fludarabine, the mutation results in a deletion of a Glutamine (Gln or Q) residue at codon 699 and a Lysine (Lys or K) residue at codon 700. When the Q699-K700 deletion is present, the mutation is a deletion of the nucleotide sequence CAGAAA corresponding to nucleotide base positions 2143 to 2148. In further cases of disclosed methods of determine the response of a subject to treatment with fludarabine, the mutation results in a substitution of a Glutamic Acid (Glu or E) residue for a Glutamine (Gln or Q) residue at codon 670. When the Q670E substitution is present, the mutation is a substitution of a Guanine (G) for a Cytosine (C) at nucleotide base position 2056.

Homo sapiens splicing factor 3b, subunit 1, 155 kDa, SF3B1, transcript variant 1, is encoded by the following mRNA sequence (NM_{—}012433.2, SEQ ID NO: 17) (portion encoding HEAT3, HEAT4, and HEAT5 domain is underlined):

| | |
|---|---|
| 1 | ggaagttctt gggagcgcca gttccgtctg tgtgttcgag tggacaaaat ggcgaagatc |
| 61 | gccaagactc acgaagatat tgaagcacag attcgagaaa ttcaaggcaa gaaggcagct |
| 121 | cttgatgaag ctcaaggagt gggcctcgat tctacaggtt attatgacca ggaaatttat |
| 181 | ggtggaagtg acagcagatt tgctggatac gtgacatcaa ttgctgcaac tgaacttgaa |
| 241 | gatgatgacg atgactattc atcatctacg agtttgcttg gtcagaagaa gccaggatat |
| 301 | catgcccctg tggcattgct taatgatata ccacagtcaa cagaacagta tgatccattt |
| 361 | gctgagcaca gacctccaaa gattgcagac cgggaagatg aatacaaaaa gcataggcgg |
| 421 | accatgataa tttccccaga gcgtcttgat ccttttgcag atggagggaa aacccctgat |
| 481 | cctaaaatga atgctaggac ttacatggat gtaatgcgag aacaacactt gactaaagaa |
| 541 | gaacgagaaa ttaggcaaca gctagcagaa aaagctaaag ctggagaact aaaagtcgtc |
| 601 | aatggagcag cagcgtccca gcctccatca aaacgaaaac ggcgttggga tcaaacagct |
| 661 | gatcagactc ctggtgccac tcccaaaaaa ctatcaagtt gggatcaggc agagacccct |
| 721 | gggcatactc cttccttaag atgggatgag acaccaggtc gtgcaaaggg aagcgagact |
| 781 | cctggagcaa ccccaggctc aaaaatatgg gatcctacac ctagccacac accagcggga |
| 841 | gctgctactc ctggacgagg tgatacacca ggccatgcga caccaggcca tggaggcgca |
| 901 | acttccagtg ctcgtaaaaa cagatgggat gaaaccccca aaacagagag agatactcct |
| 961 | gggcatggaa gtggatgggc tgagactcct cgaacagatc gaggtggaga ttctattggt |
| 1021 | gaaacaccga ctcctggagc cagtaaaaga aaatcacggt gggatgaaac accagctagt |
| 1081 | cagatgggtg gaagcactcc agttctgacc cctggaaaga caccaattgg cacaccagcc |
| 1141 | atgaacatgg ctacccctac tccaggtcac ataatgagta tgactcctga acagcttcag |
| 1201 | gcttggcggt gggaaagaga aattgatgag agaaatcgcc cactttctga tgaggaatta |
| 1261 | gatgctatgt tcccagaagg atataaggta cttcctcctc cagctggtta tgttcctatt |
| 1321 | cgaactccag ctcgaaagct gacagctact ccaacacctt tgggtggtat gactggtttc |
| 1381 | cacatgcaaa ctgaagatcg aactatgaaa agtgttaatg accagccatc tggaaatctt |
| 1441 | ccatttttaa aacctgatga tattcaatac tttgataaac tattggttga tgttgatgaa |
| 1501 | tcaacactta gtccagaaga gcaaaaagag agaaaaataa tgaagttgct tttaaaaatt |
| 1561 | aagaatggaa caccaccaat gagaaaggct gcattgcgtc agattactga taaagctcgt |
| 1621 | gaatttggag ctggtccttt gtttaatcag attcttcctc tgctgatgtc tcctacactt |
| 1681 | gaggatcaag agcgtcattt acttgtgaaa gttattgata ggatactgta caaacttgat |
| 1741 | gacttagttc gtccatatgt gcataagatc ctcgtggtca ttgaaccgct attgattgat |
| 1801 | gaagattact atgctagagt ggaaggccga gagatcattt ctaatttggc aaaggctgct |
| 1861 | ggtctggcta ctatgatctc taccatgaga cctgatatag ataacatgga tgagtatgtc |
| 1921 | cgtaacacaa cagctagagc ttttgctgtt gtagcctctg ccctgggcat tccttcttta |
| 1981 | ttgcccttct taaaagctgt gtgcaaaagc aagaagtcct ggcaagcgag acacactggt |
| 2041 | attaagattg tacaacagat agctattctt atgggctgtg ccatcttgcc acatcttaga |
| 2101 | agtttagttg aaatcattga acatggtctt gtggatgagc agcagaaagt tcggaccatc |
| 2161 | agtgctttgg ccattgctgc cttggctgaa gcagcaactc cttatggtat cgaatctttt |
| 2221 | gattctgtgt taaagccttt atggaagggt atccgccaac acagaggaaa gggtttggct |
| 2281 | gctttcttga aggctattgg gtatcttatt cctcttatgg atgcagaata tgccaactac |
| 2341 | tatactagag aagtgatgtt aatccttatt cgagaattcc agtctcctga tgaggaaatg |
| 2401 | aaaaaaattg tgctgaaggt ggtaaaacag tgttgtggga cagatggtgt agaagcaaac |
| 2461 | tacattaaaa cagagattct tcctcccttt tttaaacact tctggcagca caggatggct |
| 2521 | ttggatagaa gaaattaccg acagttagtt gatactactg tggagttggc aaacaaagta |
| 2581 | ggtgcagcag aaattatatc caggattgtg gatgatctga aagatgaagc cgaacagtac |
| 2641 | agaaaaatgg tgatggagac aattgagaaa attatgggta atttgggagc agcagatatt |
| 2701 | gatcataaac ttgaagaaca actgattgat ggtattcttt atgctttcca agaacagact |
| 2761 | acagaggact cagtaatgtt gaacggcttt ggcacagtgg ttaatgctct tggcaaacga |
| 2821 | gtcaaaccat acttgcctca gatctgtggt acagttttgt ggcgtttaaa taacaaatct |
| 2881 | gctaaagtta ggcaacaggc agctgacttg atttctcgaa ctgctgttgt catgaagact |
| 2941 | tgtcaagagg aaaaattgat gggacacttg ggtgttgtat tgtatgagta tttgggtgaa |
| 3001 | gagtaccctg aagtattggg cagcattctt ggagcactga aggccattgt aaatgtcata |
| 3061 | ggtatgcata agatgactcc accaattaaa gatctgctgc ctagactcac ccccatctta |
| 3121 | aagaacagac atgaaaaagt acaagagaat tgtattgatc ttgttggtcg tattgctgac |
| 3181 | aggggagctg aatatgtatc tgcaagagag tggatgagga tttgctttga gcttttagag |
| 3241 | ctcttaaaag cccacaaaaa ggctattcgt agagccacag tcaacacatt tggttatatt |
| 3301 | gcaaaggcca ttggccctca tgatgtattg gctacacttc tgaacaacct caaagttcaa |
| 3361 | gaaaggcaga acagagtttg taccactgta gcaatagcta ttgttgcaga aacatgttca |
| 3421 | ccctttacag tactccctgc cttaatgaat gaatacagag ttcctgaact gaatgttcaa |
| 3481 | aatggagtgt taaaatcgct ttccttcttg tttgaatata ttggtgaaat gggaaaagac |
| 3541 | tacatttatg ccgtaacacc gttacttgaa gatgctttaa tggatagaga ccttgtacac |
| 3601 | agacagacgg ctagtgcagt ggtacagcac atgtcacttg gggtttatgg atttggttgt |
| 3661 | gaagattcgc tgaatcactt gttgaactat gtatggccca atgtatttga gacatctcct |
| 3721 | catgtaattc aggcagttat gggagcccta gagggcctga gagttgctat tggaccatgt |
| 3781 | agaatgttgc aatattgttt acagggtctg tttcacccag cccggaaagt cagagatgta |
| 3841 | tattggaaaa tttacaactc catctacatt ggttcccagg acgctctcat agcacattac |
| 3901 | ccaagaatct acaacgatga taagaacacc tatattcgtt atgaacttga ctatatctta |
| 3961 | taattttatt gtttattttg tgtttaatgc acagctactt cacaccttaa acttgctttg |
| 4021 | atttggtgat gtaaactttt aaacattgca gatcagtgta gaactggtca tagaggaaga |
| 4081 | gctagaaatc cagtagcatg atttttaaat aacctgtctt tgtttttgat gttaaacagt |
| 4141 | aaatgccagt agtgaccaag aacacagtga ttatatacac tatactggag ggatttcatt |
| 4201 | tttaattcat ctttatgaag atttagaact cattccttgt gtttaaaggg aatgtttaat |
| 4261 | tgagaaataa acatttgtgt acaaaatgct aaaaaaaaaa aaaaaaaaaa aaaa |

Homo sapiens splicing factor 3b, subunit 1, 155 kDa, SF3B1, transcript variant 2, is encoded by the following mRNA sequence (NM_{—}001005526, SEQ ID NO: 18):

| | |
|---|---|
| 1 | ggaagttctt gggagcgcca gttccgtctg tgtgttcgag tggacaaaat ggcgaagatc |
| 61 | gccaagactc acgaagatat tgaagcacag attcgagaaa ttcaaggcaa gaaggcagct |
| 121 | cttgatgaag ctcaaggagt gggcctcgat tctacaggtt attatgacca ggaaatttat |
| 181 | ggtggaagtg acagcagatt tgctggatac gtgacatcaa ttgctgcaac tgaacttgaa |
| 241 | gatgatgacg atgactattc atcatctacg agtttgcttg gtcagaagaa gccaggatat |
| 301 | catgcccctg tggcattgct taatgatata ccacagtcaa cagaacagta tgatccattt |
| 361 | gctgagcaca gacctccaaa gattgcagac cgggaagatg aatacaaaaa gcataggcgg |
| 421 | accatgataa tttccccaga gcgtcttgat ccttttgcag atggcttcta ttctgctgct |
| 481 | tgaagtcaga actgctgatg gagacaaagg cacgaaagtg tacgtattcc ggattagcaa |
| 541 | cccaggaacc catcacttct gaagactcta aactgtgctg tcattttgtt tttatatgca |
| 601 | ttaaaatatt tgttttaaaa aaaaaaaaaa aaaaaaaaaa aaaaaaa |

Homo sapiens splicing factor 3b, subunit 1, 155 kDa, SF3B1, transcript variant 1, is encoded by the following amino acid sequence (NP_{—}036565.2, SEQ ID NO: 19) (portion containing HEAT3, HEAT4, and HEAT5 domain is underlined):

| | |
|---|---|
| 1 | makiakthed ieaqireiqg kkaaldeaqg vgldstgyyd qeiyggsdsr fagyvtsiaa |
| 61 | teledddddy ssstsllgqk kpgyhapval lndipqsteq ydpfaehrpp kiadredeyk |
| 121 | khrrtmiisp erldpfadgg ktpdpkmnar tymdvmreqh ltkeereirq qlaekakage |
| 181 | lkvvngaaas qppskrkrrw dqtadqtpga tpkklsswdq aetpghtpsl rwdetpgrak |
| 241 | gsetpgatpg skiwdptpsh tpagaatpgr gdtpghatpg hggatssark nrwdetpkte |
| 301 | rdtpghgsgw aetprtdrgg dsigetptpg askrksrwde tpasqmggst pvltpgktpi |
| 361 | gtpamnmatp tpghimsmtp eqlqawrwer eidernrpls deeldamfpe gykvlpppag |
| 421 | yvpirtpark ltatptplgg mtgfhmqted rtmksvndqp sgnlpflkpd diqyfdkllv |
| 481 | dvdestlspe eqkerkimkl llkikngtpp mrkaalrqit dkarefgagp lfnqilpllm |
| 541 | sptledqerh llvkvidril yklddlvrpy vhkilvviep llidedyyar vegreiisnl |
| 601 | akaaglatmi stmrpdidnm deyvrnttar afavvasalg ipsllpflka vckskkswqa |
| 661 | rhtgikivqq iailmgcail phlrslveii ehglvdeqqk vrtisalaia alaeaatpyg |
| 721 | iesfdsvlkp lwkgirqhrg kglaaflkai gyliplmdae yanyytrevm lilirefqsp |
| 781 | deemkkivlk vvkqccgtdg veanyiktei lppffkhfwq hrmaldrrny rqlvdttvel |
| 841 | ankvgaaeii srivddlkde aeqyrkmvme tiekimgnlg aadidhklee qlidgilyaf |
| 901 | qeqttedsvm lngfgtvvna lgkrvkpylp qicgtvlwrl nnksakvrqq aadlisrtav |
| 961 | vmktcqeekl mghlgvvlye ylgeeypevl gsilgalkai vnvigmhkmt ppikdllprl |
| 1021 | tpilknrhek vqencidlvg riadrgaeyv sarewmricf ellellkahk kairratvnt |
| 1081 | fgyiakaigp hdvlatllnn lkvqerqnry cttvaiaiva etcspftvlp almneyrvpe |
| 1141 | lnvqngvlks lsflfeyige mgkdyiyavt plledalmdr dlvhrqtasa vvqhmslgvy |
| 1201 | gfgcedslnh llnyvwpnvf etsphviqav mgaleglrva igpcrmlqyc lqglfhpark |
| 1261 | vrdvywkiyn siyigsqdal iahypriynd dkntyiryel dyil |

Homo sapiens splicing factor 3b, subunit 1, 155 kDa, SF3B1, transcript variant 2, is encoded by the following amino acid sequence (NP_{—}001005526, SEQ ID NO: 20):

| | |
|---|---|
| 1 | makiakthed ieaqireiqg kkaaldeaqg vgldstgyyd qeiyggsdsr fagyvtsiaa |
| 61 | teledddddy ssstsllgqk kpgyhapval lndipqsteq ydpfaehrpp kiadredeyk |
| 121 | khrrtmiisp erldpfadgf ysaa |

In some non-limiting embodiments of the disclosure, a disclosed method may be used in vitro to analyze SF3B1 sequences and sequence alterations as disclosed herein without including a act of diagnosis or medical treatment.

Other features and advantages of the disclosure will be apparent from and are encompassed by the following detailed description and claims.

### BRIEF DESCRIPTION OF THE DRAWINGS

FIG. 1 is a schematic diagram of the human SF3B1 gene (top) and protein (bottom) with its functional domains (PPP1R8 binding domain and HEAT repeats). A corresponding multiple alignment of the HEAT3, HEAT4 and HEAT5 amino acid sequences of the human SF3B1 protein with orthologous SF3B1 proteins (n=15) is provided. Amino acids conserved among species are highlighted (grey). Color-coded shapes indicate the position of the mutations found in CLL at diagnosis (green, missense mutations corresponding to K666E, K700E, and G740E; also see Table 4), in fludarabine-refractory CLL (red, missense mutations corresponding to R630S, H662Q, H662D, K666E, and K700E; in-frame deletions corresponding to delQ699_K700; also see Table 4), and in Richter syndrome (RS) (orange, missense mutations corresponding to Q670E and K700E within; also see Table 4). The following sequences are provided:

(Homo Sapiens, SEQ ID NO: 1)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(P. troglodytes, SEQ ID NO: 2)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(C. familiaris, SEQ ID NO: 3)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(B. tauris, SEQ ID NO: 4)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(M. musculus, SEQ ID NO: 5)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(R. norvegicus, SEQ ID NO: 6)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(G. gallus, SEQ ID NO: 7)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(D. rerio, SEQ ID NO: 8)
ARAFAVVASALGIPSLLPFLKAVCKSKKSWQARHTGIKIVQQIAILMGCA
ILPHLRSLVEIIEHGLVDEQQKVRTISALAIAALAEAATPYGIESFDSVL
KPLWKGIRQHRGK,
(D. melanogaster, SEQ ID NO: 9)
ARAFAVVASALGIPSLLPFLKAVCKSKKSWQARHTGIKIVQQIAILMGCA
ILPHLKALVEIIEHGLVDEQQKVRTITALAIAALAEAATPYGIESFDSVL
KPLWKGIRTHRGK,
(A. gambiae, SEQ ID NO: 10)
ARAFAVVASALGIPSLLPFLKAVCKSKKSWQARHTGIKIVQQIAILMGCA
ILPHLKSLVEIIEHGLVDEQQKVRTITALALAALAEAATPYGIESFDSVL
KPLWKGIRTHRGK,
(C. elegans, SEQ ID NO: 11)
ARAFAVVASALGIPALLPFLKAVCKSKKSWQARHTGIKIVQQMAILMGCA
VLPHLKALVDIVESGLDDEQQKVRTITALCLAALAEASSPYGIEAFDSVL
KPLWKGIRMHRGK,
(S. Pombe, SEQ ID NO: 12)
ARAFSVVASALGVPALLPFLKAVCRSKKSWQARHTGVRIIQQIALLLGCS
ILPHLKNLVDCIGHGLEDEQQKVRIMTALSLSALAEAATPYGIEAFDSVL
KPLWSGVQRHRGK,
(M. oryzae, SEQ ID NO: 13)
ARAFAVVASALGIPALLPFLQAVCRSKKSWQARHTGVKIVQQIPILMGCA
VLPHLKRLVDCIGPNLNDEQTKVRTVTSLAIAALAEAANPYGIESFDDIL
NPLWTGARKQRGK,
(M. crassa, SEQ ID NO: 14)
ARAFAVVASALGIPALLPFLRAVCRSKKSWQARHTGVKIVQQIPILMGCA
VLPHLKQLVDCIGPNLNDEQTKVRTVTSLAIAALAEASNPYGIESFDDIL
NPLWTGARKQRGK,
(A. thaliana, SEQ ID NO: 15)
ARAFSVVASALGIPALLPFLKAVCQSKRSWQARHTGIKIVQQIAILIGCA
VLPHLRSLVEIIEHGLSDENQKVRTITALSLAALAEAAAPYGIESFDSVL
KPLWKGIRSHRGK,
and
(O. Sativa, SEQ ID NO: 16)
ARAFSVVASALGTPALLPFLKAVCQSKKSWQARHTGIKIVQQIAILMGCA
VLPHLKSLVEIIEHGLSDENQKVRTITALSLATLAEAAAPYGIESFDTVL
KPLWKGIRSHRGK.

FIG. 2A-D is a series of graphs depicting the prevalence, mutual relationship with other genetic lesions, and clinical impact of SF3B1 mutations in CLL. Panel A: prevalence of SF3B1 mutations in CLL at diagnosis, in fludarabine-refractory CLL, and in Richter syndrome; numbers on top indicate the actual number of mutated samples over the total number analyzed. Panel B: mutual relationship of SF3B1 mutations with other genetic lesions in CLL at diagnosis and in fludarabine-refractory CLL. In the heat map, rows correspond to identical genes, and columns represent individual patients color-coded based on the gene status (white: wild type; red: mutations of SF3B1, mutations of NOTCH1, mutations and/or deletion of TP53, deletion of ATM). Panel C: Kaplan-Meier estimates of treatment-free survival (TFS) and overall survival (OS) from diagnosis in the consecutive series of newly diagnosed and previously untreated CLL (n=301). SF3B1 wild type (SF3B1 wt) is represented by the blue line. SF3B1 mutated cases (SF3B1 M) are represented by the red line. Panel D: Gene expression levels of BCL6, AICDA, BCL2, IRF4 and SF3B1 in normal B-cell subpopulations (Naïve; Centroblasts, CB; Centrocytes, CC; Memory) and CLL samples. Relative levels of gene expression are depicted with a color scale: red represents the highest level of expression and blue represents the lowest level.

### DETAILED DESCRIPTION OF MODES OF PRACTICING THE DISCLOSURE

The genetic lesions identified in chronic lymphocytic leukemia (CLL) do not entirely recapitulate the disease pathogenesis and the development of serious complications, such as chemorefractoriness. While investigating the coding genome of fludarabine-refractory CLL, it was discovered that mutations of SF3B1, encoding a splicing factor and representing a critical component of the cell spliceosome, were recurrent in 10/59 (17%) fludarabine-refractory cases, with a frequency significantly higher than that observed in a consecutive CLL cohort sampled at diagnosis (17/301, 5%; p=0.002). Mutations were somatically acquired, were generally represented by missense nucleotide changes, clustered in selected HEAT repeats of the SF3B1 protein, recurrently targeted three hotspots (codons 662, 666 and 700), and predicted poor prognosis. In fludarabine-refractory CLL, SF3B1 mutations and TP53 disruption distributed in a mutually-exclusive fashion (p=0.046). Identification of SF3B1 mutations indicates that splicing regulation is a novel pathogenetic mechanism of clinical relevance in CLL.

The clinical course of chronic lymphocytic leukemia (CLL) ranges from a very indolent disorder with a normal lifespan, to a rapidly progressive disease leading to death. Occasionally, CLL undergoes histological transformation to Richter syndrome (RS) (Müller-Hermelink H K et al. In: Swerdlow S H et al. eds. World Health Organization Classification of Tumours, Pathology and Genetics of Tumours of Haematopoietic and Lymphoid Tissues. Lyon, France: IARC; 2008: 180-182; Hallek M, et al. Blood. 2008; 111(12):5446-5456; Rossi D, et al. Blood. 2011; 117(12):3391-3401). The variable clinical course of CLL is driven, at least, in part, by the immunogenetic and molecular heterogeneity of the disease (Chiorazzi N, et al. N Engl J Med. 2005; 352(8):804-815).

Despite recent advances, the genetic lesions identified to date do not fully recapitulate CLL molecular pathogenesis and do not entirely explain the development of severe complications, such as chemorefractoriness, which still represent an unmet clinical need (Kay N E, et al. Leukemia 2007; 21(9):1885-1891). Fludarabine-refractoriness is due to TP53 disruption in ˜40% of refractory cases, but in a sizeable fraction of patients the molecular basis of this aggressive clinical phenotype remains unclear (Stilgenbauer S and Zenz T. Hematology Am Soc Hematol Educ Program. 2010; 2010: 481-488).

Recently, two independent studies of the CLL coding genome investigated at disease presentation have revealed a restricted number of mutated genes, including NOTCH1 (Fabbri G, et al. J Exp Med. 2011; 208(7):1389-1401; Puente X S, et al. Nature. 2011; 475(7354):101-105). These studies have provided a proof of concept that, similar to other malignancies, genome-wide mutational analysis might identify novel lesions of potential biological and clinical relevance in CLL. Following initial findings from whole exome sequencing of the coding genome of fludarabine-refractory CLL, the occurrence of recurrent mutations of SF3B1, a critical component of the cell spliceosome, is disclosed herein.

The terms “nucleic acid” and “polynucleotide” are used interchangeably herein to refer to single- or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. This refers to the nucleic acid as originally isolated and does not exclude genes or sequences later added to the isolated nucleic acid by the hand of man. In addition to preparation of nucleic acid molecules as described above, the disclosure includes preparation of nucleic acid molecules by direct or indirect immobilization on a solid support or solid phase medium. Direct immobilization may be mediated by hydrogen bonds, such as in the case of hybridization as a non-limiting example, or be mediated by one or more covalent bonds. Non-limiting examples include hybridization of nucleic acid molecules to a polynucleotide probe on a microarray or a bead or another solid support to detect a nucleic acid molecule of interest. Optionally, the hybridized nucleic acid molecules may be those amplified by PCR. Indirect immobilization of a nucleic acid molecule may be mediated by binding to an immobilized polymerase, such as an RNA polymerase or DNA polymerase. In additional embodiments, a nucleic acid molecule may be prepared for sequencing by ligation to a known nucleic acid sequence or binding to a primer polynucleotide by basepair complementarity. In some embodiments, an immobilized nucleic acid molecule may be sequenced without need for amplification or replication. In further embodiments, a prepared nucleic acid molecule may be an RNA molecule that has been detectably labeled to aid in its analysis or an RNA molecule that has been coverted into a cDNA molecule for use as described herein.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

An “isolated polypeptide” is one that has been identified and separated and/or recovered from a component of its natural environment. In some embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., protein subunit) derived from nature (e.g., from any species). Such native sequence polynucleotides and polypeptides can be isolated from nature or can be produced by recombinant or synthetic means.

A “mutant or mutated” polynucleotide, as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the disclosure and evaluating one or more biological activities of the encoded polypeptide as described herein and/or using any of a number of techniques well known in the art. “Mutant” polynucleotides of the disclosure contain one or more substitutions, deletions, additions and/or insertions that alter the function of the resultant polypeptide encoded therein. Alternatively, or in addition, “modified” polynucleotides of the disclosure contain one or more substitutions, deletions, additions and/or insertions that do not alter the function of the resultant polypeptide encoded therein. In some embodiments, a “mutant or mutated” polynucleotide is defined by reference to a wildtype sequence as disclosed herein. Additionally, a “mutant or mutated” polynucleotide may be prepared, and optionally detected, in the same manner as other polynucleotides disclosed herein.

A “mutant or mutated” polypeptide, as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the disclosure and evaluating one or more biological activities of the polypeptide as described herein and/or using any of a number of techniques well known in the art. “Mutant” polypeptides of the disclosure contain one or more substitutions, deletions, additions and/or insertions that alter the function of the resultant polypeptide. Alternatively, or in addition, “modified” polypeptides of the disclosure contain one or more substitutions, deletions, additions and/or insertions that do not alter the function of the resultant polypeptide. In some embodiments, a “mutant or mutated” polypeptide is defined by reference to a wildtype sequence as disclosed herein. Additionally, a “mutant or mutated” polypeptide may be analyzed or detected by any method known to the skilled person. Non-limiting examples include peptide sequencing, analysis by mass spectroscopy, and binding by antibodies or receptors.

Modifications may be made in the structure of the wild type or mutant polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant or portion of a polypeptide of the disclosure, one skilled in the art will typically change one or more of the codons of the encoding DNA sequence.

For example, certain amino acids may be substituted for other amino acids in a protein structure without appreciable loss of its ability to bind other polypeptides or cells. Because it is the binding capacity and nature of a protein that defines that the biological functional activity of a protein, certain amino acid sequence substitutions can be made in a protein sequence, and, of course, the underlying DNA coding sequence of the protein, and nevertheless obtain a protein with like properties. It is thus contemplated that various changes may be made in the peptide sequences of the disclosed compositions, or corresponding DNA sequences that encode said peptides without appreciable loss of their biological utility or activity.

In many instances, a modified polypeptide will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A modified polypeptide may also, or alternatively, contain nonconservative changes. In a preferred embodiment, variant polypeptides differ from a native sequence by substitution, deletion or addition of five amino acids or fewer. Modified polypeptides may also (or alternatively) be modified by, for example, the deletion or addition of amino acids that have minimal influence on the immunogenicity, secondary structure and hydropathic nature of the polypeptide.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to a biotin, streptavidin, or Fc immunoglobulin.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One non-limiting example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

“Homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least 70%, at least 75%, at least 80%, at least 90%, at least 95%, at least 98%, or at least 99% polynucleotide or polypeptide homology with a polynucleotide or polypeptide described herein.

It will be appreciated by those of ordinary skill in the art that, as a result of the degeneracy of the genetic code, there are multiple nucleotide sequences that encode a polypeptide as described herein. Some of these polynucleotides bear minimal homology to the nucleotide sequence of any native gene. Nonetheless, polynucleotides that encode a polypeptide of the present disclosure but which vary due to differences in codon usage are specifically contemplated by the disclosure. Further, alleles of the genes including the polynucleotide sequences provided herein are within the scope of the disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In certain embodiments of the present disclosure, mutagenesis of the disclosed polynucleotide sequences is performed in order to alter one or more properties of the encoded polypeptide, such as its binding specificity or binding strength. Techniques for mutagenesis are well-known in the art, and are widely used to create variants of both polypeptides and polynucleotides. A mutagenesis approach, such as site-specific mutagenesis, is employed for the preparation of variants and/or derivatives of the polypeptides described herein. By this approach, specific modifications in a polypeptide sequence are made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences include the nucleotide sequence of the desired mutation, as well as a sufficient number of adjacent nucleotides, to provide a primer sequence of sufficient size and sequence complexity to form a stable duplex on both sides of the deletion junction being traversed. Mutations are employed in a selected polynucleotide sequence to improve, alter, decrease, modify, or otherwise change the properties of the polynucleotide itself, and/or alter the properties, activity, composition, stability, or primary sequence of the encoded polypeptide.

In other embodiments of the present disclosure, the polynucleotide sequences provided herein are used as probes or primers for nucleic acid hybridization, e.g., as PCR primers. The ability of such nucleic acid probes to specifically hybridize to a sequence of interest enables them to detect the presence of complementary sequences in a given sample. However, other uses are also encompassed by the disclosure, such as the use of the sequence information for the preparation of mutant species primers, or primers for use in preparing other genetic constructions. As such, nucleic acid segments of the disclosure that include a sequence region of at least about a 15-nucleotide long contiguous sequence that has the same sequence as, or is complementary to, a 15 nucleotide long contiguous sequence disclosed herein is particularly useful. Longer contiguous identical or complementary sequences, e.g., those of about 20, 30, 40, 50, 100, 200, 500, 1000 (including all intermediate lengths) including full length sequences, and all lengths in between, are also used in certain embodiments. The disclosure thus includes use of disclosed sequences in the design and preparation of nucleic acid primers and probes, such as for use in nucleic acid amplification and detection as non-limiting examples. In some embodiments, the primers may be used for nucleic acid sequencing to detect a disclosed sequence, such as a mutant sequence.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting, and/or primers for use in, e.g., polymerase chain reaction (PCR), quantitative PCR, or real-time PCR. The total size of fragment, as well as the size of the complementary stretch(es), ultimately depends on the intended use or application of the particular nucleic acid segment. Smaller fragments are generally used in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 12 bases in length are generally preferred, though, in order to increase stability and selectivity of the hybrid, and thereby improve the quality and degree of specific hybrid molecules obtained. Nucleic acid molecules having gene-complementary stretches of 15 to 25 contiguous nucleotides, or even longer where desired, are generally preferred.

Hybridization probes are selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences is governed by various factors. For example, one may wish to employ primers from towards the termini of the total sequence.

Polynucleotide of the present disclosure, or fragments or modified sequences thereof, are readily prepared by, for example, directly synthesizing the fragment by chemical means, as is commonly practiced using an automated oligonucleotide synthesizer. Also, fragments are obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology.

In some embodiments, polynucleotides as disclosed herein may be prepared for sequence determination or detection by any method known to the skilled person. Non-limiting examples include sequencing based on 1) reversible dye-terminators and attachment of DNA molecules to primers on a slide with amplification using four types of reversible terminator bases to extend the DNA only one nucleotide at a time followed by removal of the dye along with the terminal 3′ blocker to allow the next cycle of extension; 2) ligation of immobilized oligonucleotides of known sequences followed by PCR (optionally emulsion PCR) and sequencing; 3) hydrogen ion release due to nucleotide extension with detection by semiconductor; 4) nanoball sequencing; 5) addition of polyA tail adapters followed by nucleotide extension as sequencing; 6) single molecule real time (SMRT) sequencing by use of immobilized polymerase; 7) massively parallel signature sequencing (MPSS); 8) Polony sequencing; 9) pyrosequencing via single DNA templates hybridized to single primer coated beads; and 10) RNA polymerase (RNAP) mediated sequencing.

The analyses of the disclosure may be preceded or followed by a variety of related actions. In some embodiments, an analysis is preceded by a determination or diagnosis of a human subject as in need of the analysis. The analysis may be preceded by a determination of a need for the analysis, such as that by a medical doctor, nurse or other health care provider or professional, or those working under their instruction, or personnel of a health insurance or maintenance organization in approving the performance of the measurement as a basis to request reimbursement or payment for the performance. In some embodiments, an analysis may be followed by payment for performance of a disclosed method.

The analyses of the disclosure may also be preceded by preparatory acts necessary to an actual analysis. Non-limiting examples include the actual obtaining of a cell containing or nucleic acid containing or polypeptide containing sample from a human subject; or receipt of such a sample; or sectioning a cell containing sample; or isolating cells from a cell containing sample; or preparing nucleic acid molecules from cells of a cell containing sample; or reverse transcribing RNA from cells of a cell containing sample.

The disclosure further provides kits for the practice of any disclosed method as described herein. A kit will typically comprise one or more reagents to detect nucleic acid sequence or polypeptide sequence as described herein for the practice of the present disclosure. Non-limiting examples include polynucleotide probes or primers for the detection of expression levels, one or more enzymes used in the methods of the disclosure, and one or more containers or solid supports or solid medium for use in the practice of the disclosure. In some embodiments, the kit will include an array or other solid media, including a bead as a non-limiting example, for the detection of sequences as described herein. In other embodiments, the kit may comprise one or more antibodies that are immunoreactive with epitopes present on a polypeptide which indicates the presence of a gene sequence alteration as disclosed herein. In some embodiments, the antibody will be an antibody fragment.

A kit of the disclosure may also include instructional materials disclosing or describing the use of the kit or a primer or probe of the present disclosure in a method of the disclosure as provided herein. A kit may also include additional components to facilitate the particular application for which the kit is designed. Thus, for example, a kit may additionally contain means of detecting the label (e.g. enzyme substrates for enzymatic labels, filter sets to detect fluorescent labels, appropriate secondary labels such as a sheep anti-mouse-HRP, or the like). A kit may additionally include buffers and other reagents recognized for use in a method of the disclosure. In some embodiments, a kit may be designed for use as an in vitro diagnostic.

Having now generally provided the disclosure, the same will be more readily understood through reference to the following examples which are provided by way of illustration, and are not intended to be limiting of the disclosure, unless specified.

### EXAMPLES

### Example 1

### Patients and Methods

### Patients

The study population included three clinical cohorts representative of different disease phases: i) fludarabine-refractory CLL (n=59), including cases (n=11) subjected to whole exome sequencing (Table 1); ii) a consecutive series of newly diagnosed and previously untreated CLL (n=301) (Table 2); and iii) clonally related RS (n=33; all diffuse large B cell lymphomas) (Table 3). CLL diagnosis was based on the IWCLL-NCI criteria (Hallek M, et al. Blood. 2008; 111(12):5446-5456); diagnosis of fludarabine-refractoriness was according to guidelines (Hallek M, et al. Blood. 2008; 111(12):5446-5456); RS was based on histological criteria (Müller-Hermelink H K, et al. Swerdlow S H et al eds. World Health Organization Classification of Tumours, Pathology and Genetics of Tumours of Haematopoietic and Lymphoid Tissues. Lyon, France: IARC; 2008: 180-182; Stein H et al. Swerdlow S H et al. eds. World Health Organization Classification of Tumours, Pathology and Genetics of Tumours of Haematopoietic and Lymphoid Tissues. Lyon, France: IARC; 2008: 233-237). Peripheral blood tumor samples were obtained as follows: i) for fludarabine-refractory CLL, immediately before starting the treatment to which the patient eventually failed to respond; ii) for newly diagnosed and previously untreated CLL, at disease presentation. All RS studies were performed on RS diagnostic biopsies. Normal DNAs from the same patients were obtained from saliva or from purified granulocytes and confirmed to be tumor-free by PCR of tumor-specific IGHV-D-J rearrangements. Patients provided informed consent in accordance with local IRB requirements and the Declaration of Helsinki. The study was approved by the Ethical Committee of the Ospedale Maggiore della Caritá di Novara associated with the Amedeo Avogadro University of Eastern Piedmont (Protocol Code 59/CE; Study Number CE 8/11).

TABLE 1
Clinical and biological characteristics of the fludarabine-refractory
CLL cohort^{a}

| | | | SF3B1 | | | | |
|---|---|---|---|---|---|---|---|
| | All | | mutated | | SF3B1 wt | | |
| | (n = 59) | | (n = 10) | | (n = 49) | | |
| | Number | % | Number | % | Number | % | p |
| Age >65 years | 37 | 62.7 | 6 | 60.0 | 31 | 63.0 | 1.000 |
| Male | 40 | 67.8 | 7 | 70.0 | 33 | 67.3 | 1.000 |
| Rai stage III-IV | 28 | 47.5 | 7 | 70.0 | 21 | 42.9 | .168 |
| Number of | | | | | | | .264 |
| prior therapies | | | | | | | |
| 0 | 26 | 44.1 | 4 | 40.0 | 22 | 44.9 | |
| 1 | 24 | 40.7 | 6 | 60.0 | 18 | 36.7 | |
| >1 | 9 | 15.3 | 0 | 0 | 9 | 18.4 | |
| Treatment | | | | | | | .750 |
| regimen at | | | | | | | |
| refractoriness | | | | | | | |
| FCR | 17 | 28.8 | 4 | 40.0 | 13 | 26.5 | |
| FR | 3 | 5.1 | 0 | 0 | 3 | 6.1 | |
| FC | 19 | 32.2 | 2 | 20.0 | 17 | 34.7 | |
| F | 20 | 33.8 | 4 | 40.0 | 16 | 32.7 | |
| IGHV | 48 | 81.4 | 8 | 80.0 | 40 | 81.6 | 1.000 |
| identity ≧98% | | | | | | | |
| CD38 ≧30% | 34 | 57.6 | 6 | 60.0 | 28 | 57.1 | 1.000 |
| ZAP70 ≧20% | 39 | 66.1 | 6 | 60.0 | 33 | 67.3 | .721 |
| TP53 | 23 | 39.0 | 1 | 10.0 | 22 | 44.9 | .072 |
| disruption | | | | | | | |
| NOTCH1 | 14 | 23.7 | 1 | 10.0 | 13 | 26.5 | .425 |
| mutations | | | | | | | |
| 11q22-q23 | 15 | 25.4 | 3 | 30.0 | 12 | 24.5 | .704 |
| deletion | | | | | | | |
| Trisomy 12 | 16 | 27.1 | 0 | 0 | 16 | 32.7 | .049 |
| 13q14 deletion | 31 | 52.5 | 6 | 60.0 | 25 | 51.1 | .734 |
| Normal FISH | 10 | 16.9 | 5 | 50.0 | 5 | 10.2 | .008 |

^{a}wt, wild type; FCR, fludarabine, cyclophosphamide, rituximab; FR, fludarabine, rituximab; FC, fludarabine, cyclophosphamide; F, fludarabine; IGHV, immunoglobulin heavy variable gene; FISH, fluorescence in situ hybridization

TABLE 2
Clinical and biological characteristics of the consecutive series of newly
diagnosed and previously untreated CLL^{a}

| | All | | SF3B1 mutated | | SF3B1 wt | | |
|---|---|---|---|---|---|---|---|
| | Number | % | Number | % | Number | % | p |
| Age >65 years | 183/301 | 60.8 | 13/17 | 76.5 | 170/284 | 59.9 | .173 |
| Male | 163/301 | 54.2 | 13/17 | 76.5 | 150/284 | 52.8 | .057 |
| Rai stage III-IV | 33/301 | 11.0 | 7/17 | 41.2 | 26/284 | 9.2 | .001 |
| IGHV identity ≧98% | 100/294 | 34.0 | 8/17 | 47.1 | 92/277 | 33.2 | .242 |
| CD38 ≧30% | 81/298 | 27.2 | 7/17 | 41.2 | 74/281 | 26.3 | .259 |
| ZAP70 ≧20% | 77/253 | 30.0 | 8/13 | 61.5 | 69/240 | 28.7 | .025 |
| TP53 disruption | 30/301 | 10.0 | 1/17 | 5.9 | 29/284 | 10.2 | 1.000 |
| NOTCH1 mutations | 34/301 | 11.3 | 1/17 | 5.9 | 33/284 | 11.6 | .704 |
| 11q22-q23 deletion | 21/301 | 7.0 | 2/17 | 11.8 | 19/284 | 6.7 | .336 |
| Trisomy 12 | 58/301 | 19.3 | 1/17 | 5.9 | 57/284 | 20.1 | .211 |
| 13q14 deletion | 157/301 | 52.2 | 8/17 | 47.1 | 149/284 | 52.5 | .665 |
| Normal FISH | 89/301 | 29.6 | 8/17 | 47.1 | 81/284 | 28.5 | .104 |

^{a}wt, wild type; IGHV, immunoglobulin heavy variable gene; FISH, fluorescence in situ hybridization

TABLE 3
Clinical and biological characteristics of the RS cohort^{a}

| | Number (n = 33) | % |
|---|---|---|
| Clinical features at RS diagnosis | | |
| Age >65 years | 19 | 57.6 |
| Male | 22 | 66.7 |
| ECOG PS >1 | 13 | 39.3 |
| Ann Arbor stage III-IV | 33 | 100 |
| Rai stage III-IV | 14 | 42.4 |
| B symptoms | 13 | 39.3 |
| Tumor size >5 cm | 24 | 72.7 |
| Platelets <100 × 10⁹/L | 7 | 21.2 |
| LDH >1.5 ULN | 17 | 51.5 |
| Prior CLL therapies >1 | 7 | 21.2 |
| Pathologic features at RS diagnosis | | |
| Non-GC phenotype | 32 | 96.9 |
| EBV infection | 0 | 0 |
| Genetic features | | |
| TP53 disruption | 18 | 54.5 |
| c-MYC aberrations | 5 | 15.1 |
| NOTCH1 mutations | 13 | 39.4 |
| IGHV identity ≧98% | 24 | 72.7 |

^{a}ULN, upper limit of normal; GC, germinal center; IGHV, immunoglobulin heavy variable gene

### Mutation Analysis of SF3B1

Mutational analysis of SF3B1 (exons 1-25, including splicing sites; RefSeq or Genbank Accession No. NM_{—}012433.2) was performed on PCR amplimers obtained from genomic DNA by a combination of Sanger sequencing (performed on an ABI PRISM 3100 Genetic Analyzer, Applied Biosystems) and targeted next generation sequencing (performed on a Genome Sequencer Junior, 454 Life Sciences, Roche, Branford, Conn.; mean coverage ˜200×). Sanger sequences were compared to the corresponding germline RefSeq using Mutation Surveyor Version 2.41 (SoftGenetics, State College, Pa.) after both automated and manual curation. Sequencing reads obtained by next generation sequencing were mapped on RefSeq using the Amplicon Variant Analyzer software package (Roche). All sequence variants identified by Sanger sequencing or next generation sequencing were subsequently confirmed by Sanger sequencing from both strands on independent amplimers. Synonymous mutations, germline polymorphisms known from databases (dbSNP132, Ensembl Database, UCSC Genome Browser), and changes present in matched normal DNA were removed from the analysis. Molecular studies were performed in blind with respect to clinical data. The prediction of functional effects of the amino acid substitutions was performed by using the PolyPhen-2 algorithm (Software version 2.1, genetics.bwh.harvard.edu/pph2) (Adzhubei I A, et al. Nat Methods. 2010; 7(4)248-249).

### Analysis of FISH Karyotype and of IGHV, TP53 and NOTCH1 Mutations

FISH analysis was performed as reported using probes LSI13 and LSID13S319, CEP12, LSIp53, and LSIATM (Abbott, Rome, Italy) (Rossi D, et al. Clin Cancer Res. 2009; 15(3):995-1004). IGHV mutational status was investigated as previously reported (Rossi D, et al. Clin Cancer Res. 2009; 15(13):4415-4422). Sequences were aligned to the ImMunoGeneTics sequence directory and considered mutated if identity to corresponding germline genes was <98% (Hamblin T J, et al. Blood. 1999; 94(6):1848-1854; Damle R N, et al. Blood. 1999; 94(6):1840-1847). TP53 and NOTCH1 mutations were analyzed as reported (Rossi D, et al. Clin Cancer Res. 2009; 15(3):995-1004; Fabbri G, et al. J Exp Med. 2011; 208(7):1389-1401).

### Copy Number Analysis

Genome-wide DNA profiles were obtained from high molecular weight genomic DNA of CLL patients using the Affymetrix Genome-Wide Human SNP Array 6.0 (Affymetrix, Santa Clara, Calif., USA), following the manufacturer's instructions. The bioinformatics pipeline used for the identification of copy number alterations was previously described (Pasqualucci L, et al. Nat Genet. 2011; doi: 10.1038/ng.892; Rinaldi A, et al. Br J Haematol. 2011; doi: 10.1111/j.1365-2141.2011.08789.x).

### Gene Expression Profile Analysis

Gene expression profile analysis of purified normal B cell subpopulations and CLL samples was performed using Affymetrix HG-U133_plus2 arrays as part of an independent study (GEO database GSE12195). The probes used in FIG. 2D are the following: 228758_at, 203140_at, and 215990_s_at (for BCL6); 219841_at and 224499_s_at (for AICDA); 203684_s_at and 203685_at (for BCL2); 204562_at and 216986_s_at (for IRF4); and 201070_x_at, 201071_x_at, 211185_s_at, and 214305_s_at (for SF3B1).

### Statistical Analysis

Overall survival was measured from date of diagnosis to date of death (event) or of last follow-up (censoring). Treatment free survival was measured from date of diagnosis to date of progressive and symptomatic disease requiring treatment according to IWCLL-NCI guidelines (event), death, or last follow up (censoring) (Hallek M, et al. Blood. 2008; 111(12):5446-5456). Survival was estimated by the Kaplan-Meier method (Kaplan E L and Meier P. Am Stat Assoc. 1958; 53:457-481). The crude association between SF3B1 mutations and survival was estimated by log-rank analysis (Kaplan E L and Meier P. Am Stat Assoc. 1958; 53:457-481).

Categorical variables were compared by chi-square test and exact tests when appropriate. All statistical tests were two-sided. Statistical significance was defined as p value<0.05. The analysis was performed with the Statistical Package for the Social Sciences (SPSS) software v.18.0 (Chicago, Ill.).

### Example 2

### Mutations in the SF3B1 Splicing Factor Affect Progression and Fludarabine-Refractoriness of Chronic Lymphocytic Leukemia

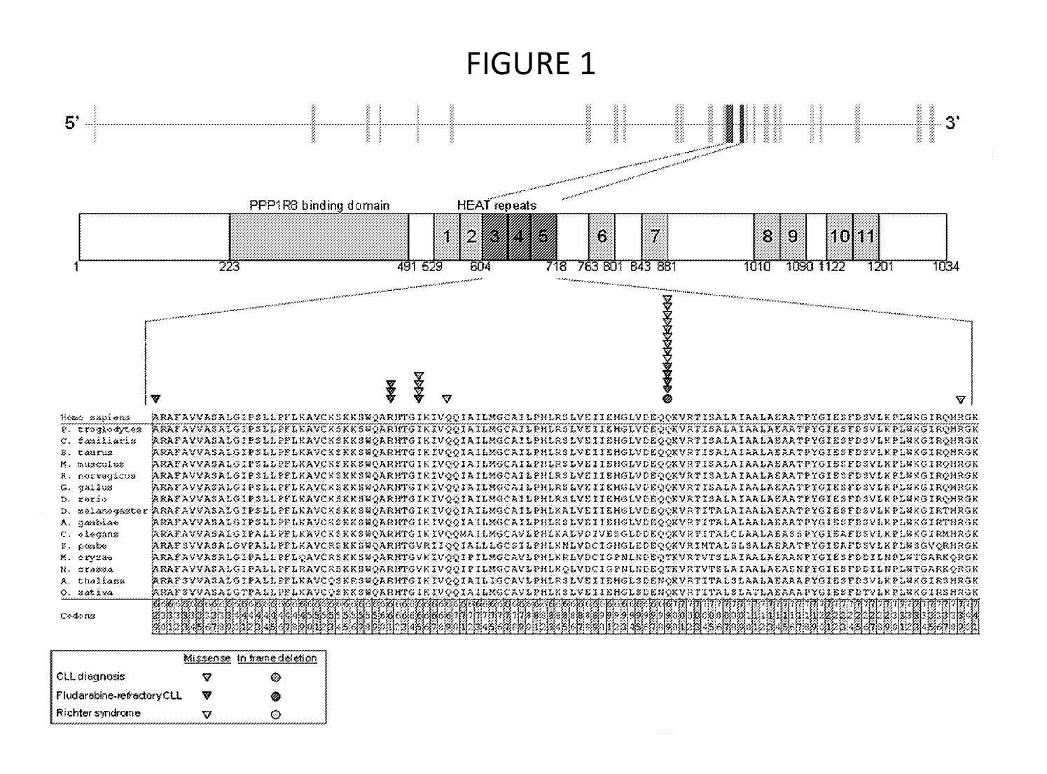

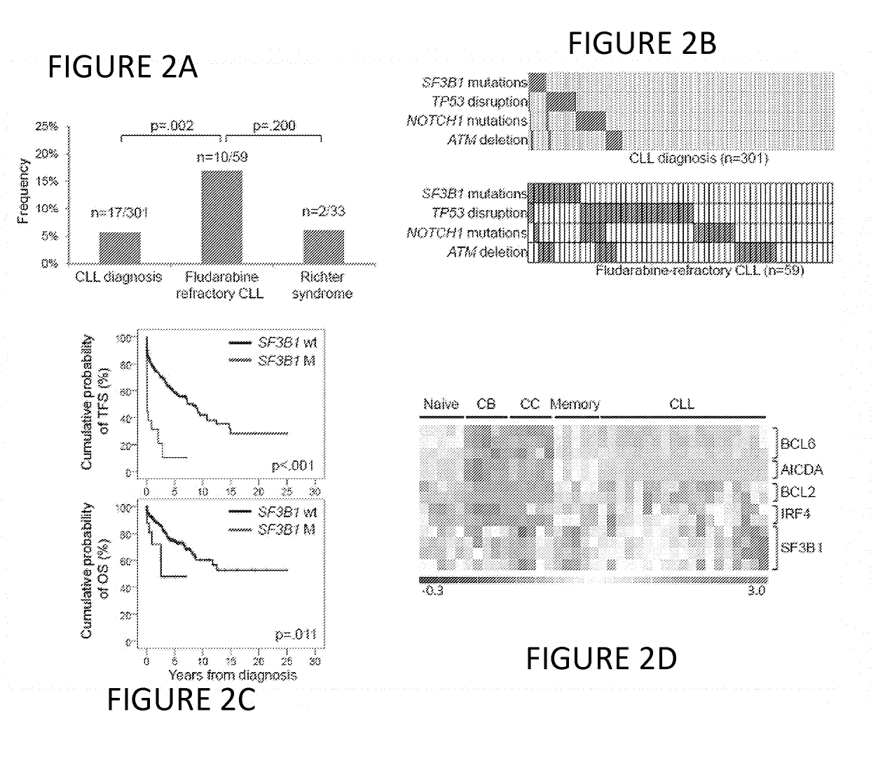

Following the initial observation of recurrent SF3B1 mutations in 3/11 fludarabine-refractory CLL analyzed by whole exome sequencing, targeted re-sequencing of the SF3B1 coding sequence and splice sites was performed in 48 additional cases of progressive and fludarabine-refractory CLL (total number of cases analyzed: 59), collected at the time of progression immediately before starting the treatment to which the patient eventually failed to respond (Table 1). SF3B1 was altered in 10/59 (17%) fludarabine-refractory CLL by missense mutations (n=9) or in-frame deletions (n=1) clustering in the HEAT3, HEAT4 and HEAT5 repeats of the SF3B1 protein (FIG. 1 and FIG. 2A; Table 4). Two sites that are highly conserved inter-species (codon 662 and codon 700) were recurrently mutated in 3 and 5 cases, respectively (FIG. 1). SF3B1 mutations were monoallelic and were predicted to be functionally significant according to the PolyPhen-2 algorithm (Table 4) (Adzhubei I A, et al. Nat Methods. 2010; 7(4):248-249). These data document that mutations of SF3B1, a splicing factor that is a critical component of the spliceosome, recurrently associate with fludarabine-refractory CLL.

The biological characteristics of fludarabine-refractory CLL harboring SF3B1 mutations are summarized in Table 1. Mutations occurred irrespective of the IGHV mutation status, CD38 expression and ZAP70 expression. At the time of fludarabine-refractoriness, SF3B1 mutations were enriched in cases harboring a normal FISH karyotype (p=0.008; Table 1). Also, SF3B1 mutations distributed in a mutually exclusive fashion compared to TP53 disruption tested by deletion and/or mutation (mutual information I=0.0609; p=0.046; FIG. 2B). By combining SF3B1 mutations with other genetic lesions enriched in chemorefractory cases (TP53 disruption, NOTCH1 mutations, ATM deletion) (Fabbri G, et al. J Exp Med. 2011; 208(7):1389-1401; Döhner H, et al. N Engl J Med. 2000; 343(26):1910-1916; Rossi D, et al. Clin Cancer Res. 2009; 15(3):995-1004; Zenz T, et al. Blood. 2009; 114(13):2589-2597; Stilgenbauer S, et al. J Clin Oncol. 2009; 27(24):3994-4001), fludarabine-refractory CLL appeared to be characterized by multiple molecular alterations that, to some extent, are mutually exclusive (FIG. 2B).

To investigate whether SF3B1 mutations are restricted to chemorefractory cases, the prevalence of mutations observed at the time of fludarabine-refractoriness was then compared to the prevalence of mutations observed in other disease phases. In a consecutive series evaluated at CLL diagnosis, SF3B1 mutations were rare (17/301; 5%) (FIG. 2A; Table 4), and showed a crude association with short treatment free survival (p<0.001) and overall survival (p=0.011) (FIG. 2C). Remarkably, 5/17 (29%) CLL mutated at diagnosis were primary fludarabine-refractory patients. One patient with wild type SF3B1 alleles at diagnosis subsequently acquired a SF3B1 mutation concomitant with the development of fludarabine-refractoriness (case 7915 in Table 4). In CLL investigated at diagnosis, the hot-spot distribution and molecular spectrum of SF3B1 mutations, as well as their mutual relationship with other genetic lesions, were similar to those observed in fludarabine-refractory CLL (FIGS. 1 and 2B; Table 4). SF3B1 mutations were only found in 2/33 (6.0%) clonally-related RS (FIGS. 1 and 2A; Table 4). Across the different disease phases investigated, mutations were confirmed to be somatically acquired in all cases (n=18) for which germline DNA was available (Table 4). Although the relative expression of SF3B1 in CLL was higher compared to normal B-cell subsets (FIG. 2D), extensive investigation by SNP array analysis ruled out focal copy number abnormalities of SF3B1 in this leukemia (n=0/323). These data document that SF3B1 mutations: i) occur at a low rate at CLL presentation, whereas they are enriched in fludarabine-refractory cases; ii) play a minor role in RS transformation, corroborating the notion that CLL histologic shift is molecularly distinct from chemorefractory progression without RS transformation (Rossi D, et al. Blood. 2011; 117(12):3391-3401).

The identification of SF3B1 mutations points to the involvement of splicing regulation as a novel pathogenetic mechanism in CLL. SF3B1 is a critical component of both major (U2-like) and minor (U12-like) spliceosomes (Luke M M, et al. Mol Cell Biol. 1996; 16(6):2744-2755; Wang C, et al. Genes Dev. 1998; 12(10):1409-1414; Das B K, et al. Mol Cell Biol. 1999; 19(10):6796-6802), which enact the precise excision of introns from pre-mRNA (Wahl M C, et al. Cell. 2009; 136(4):701-718; David C J and Manley J L. Genes Dev. 2010; 24(21):2343-2364; Ward A J and Cooper T A. J Pathol. 2010; 220(2):152-163. The precise biological role of SF3B1 mutations in CLL is currently elusive. The pathogenicity of SF3B1 mutations in CLL is strongly supported by the clustering of these mutations in evolutionarily conserved hotspots localized within HEAT domains, which are tandemly arranged curlicue-like structures serving as flexible scaffolding on which other components can assemble (Andrade M A and Bork P. Nat Genet. 1995; 11(2):115-116; Andrade M A, et al. J Struct Biol. 2001; 134(2-3):117-131). Also, the observation that SF3B1 regulates the alternative splicing program of genes controlling cell cycle progression and apoptosis points to a potential contribution of SF3B1 mutations in modulating tumor cell proliferation and survival (David C J and Manley J L. Genes Dev. 2010; 24(21):2343-2364; Kaida D, et al. Nat Chem Biol. 2007; 3(9):576-583; Corrionero A, et al. Genes Dev. 2011; 25(5):445-459).

In addition to pathogenetic implications, SF3B1 mutations also provide a therapeutic target for SF3B1 inhibitors (Kaida D, et al. Nat Chem Biol. 2007; 3(9):576-583; Corrionero A, et al. Genes Dev. 2011; 25(5):445-459), which are currently under pre-clinical development as anti-cancer drugs.

TABLE 4
SF3B1 mutations in CLL and RS

| | | | | Affected | Conserved | | | COSMIC |
|---|---|---|---|---|---|---|---|---|
| Sample ID | Disease phase | Nucleotide change^{c} | Amino acid change^{d} | domain | site^{e} | PolyPhen-2^{f} | Score | v54^{g} |
| 7040^{a} | CLL diagnosis | c.2044A > G | p.K666E | HEAT4 | No | Damaging | 1.000 | No |
| 11772^{a} | CLL diagnosis | c.2044A > G | p.K666E | HEAT4 | No | Damaging | 1.000 | No |
| 9094 | CLL diagnosis | c.2046G > T | p.K666N | HEAT4 | No | Damaging | 1.000 | No |
| 4602^{a} | CLL diagnosis | c.2146A > G | p.K700E | HEAT5 | Yes | Damaging | 1.000 | Yes |
| 4681^{a} | CLL diagnosis | c.2146A > G | p.K700E | HEAT5 | Yes | Damaging | 1.000 | Yes |
| 7561^{a} | CLL diagnosis | c.2146A > G | p.K700E | HEAT5 | Yes | Damaging | 1.000 | Yes |
| 10676^{a} | CLL diagnosis | c.2146A > G | p.K700E | HEAT5 | Yes | Damaging | 1.000 | Yes |
| 11196^{a} | CLL diagnosis | c.2146A > G | p.K700E | HEAT5 | Yes | Damaging | 1.000 | Yes |
| 11197^{a} | CLL diagnosis | c.2146A > G | p.K700E | HEAT5 | Yes | Damaging | 1.000 | Yes |
| 11489^{a} | CLL diagnosis | c.2146A > G | p.K700E | HEAT5 | Yes | Damaging | 1.000 | Yes |
| 11785^{a} | CLL diagnosis | c.2146A > G | p.K700E | HEAT5 | Yes | Damaging | 1.000 | Yes |
| 3950^{a} | CLL diagnosis | c.2267G > A | p.G740E | — | Yes | Damaging | 0.949 | No |
| 4845^{a} | Fludarabine-refractory CLL^{b} | c.1938A > T | p.R630S | HEAT3 | Yes | Damaging | 1.000 | No |
| 7425^{a} | Fludarabine-refractory CLL^{b} | c.2034C > A | p.H662Q | HEAT4 | Yes | Damaging | 1.000 | No |
| 7228 | Fludarabine-refractory CLL^{b} | c.2034C > A | p.H662Q | HEAT4 | Yes | Damaging | 1.000 | No |
| 12627 | Fludarabine-refractory CLL | c.2032C > G | p.H662D | HEAT4 | Yes | Damaging | 1.000 | No |
| 7915^{a} | Fludarabine-refractory CLL | c.2044A > G | p.K666E | HEAT4 | No | Damaging | 1.000 | No |
| 12571 | Fludarabine-refractory CLL | c.2146A > G | p.K700E | HEAT5 | Yes | Damaging | 1.000 | Yes |
| 12631 | Fludarabine-refractory CLL | c.2146A > G | p.K700E | HEAT5 | Yes | Damaging | 1.000 | Yes |
| 14220_R^{a} | Fludarabine-refractory CLL | c.2146A > G | p.K700E | HEAT5 | Yes | Damaging | 1.000 | Yes |
| 3981^{a} | Fludarabine-refractory CLL^{b} | c.2146A > G | p.K700E | HEAT5 | Yes | Damaging | 1.000 | Yes |
| 5565^{a} | Fludarabine-refractory CLL^{b} | c.2143_2148delCAGAAA | p.delQ699_K700 | HEAT5 | Yes | na | na | No |
| 8343 | Richter syndrome | c.2056C > G | p.Q670E | HEAT4 | Yes | Damaging | 0.999 | No |
| 7509^{a} | Richter syndrome | c.2146A > G | p.K700E | HEAT5 | Yes | Damaging | 1.000 | Yes |

^{a}For these patients, paired normal DNA was available and confirmed the somatic origin of the mutation
^{b}In these patients, the time of fludarabine-refractoriness was concomitant with clinical diagnosis
^{c}Numbering according to GenBank accession No. NM_012433.2
^{d}Numbering according to GenBank accession No. NP_036565.2
^{e}Position conserved among SF3B1 orthologues
^{f}na, not applicable, since the PolyPhen-2 algorithm predicts only the impact of amino acid substitutions
^{g}Mutations listed in the Catalog of Somatic Mutations in Cancer (COSMIC) database v54 release (http://www.sanger.ac.uk/genetics/CGP/cosmic/)

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of prognosing a subject with chronic lymphocytic leukemia (CLL), comprising,
(a) obtaining a biological sample from the subject;
(b) determining the sequence of a portion of the SF3B1 gene in the sample, wherein the portion of the SF3B1 gene comprises a sequence that encodes for a HEAT3, HEAT4 or HEAT5 domain;
(c) analyzing the sequence of the SF3B1 gene for a mutation,
wherein the presence of the mutation in the sequence of the SF3B1 gene predicts a decreased survival of the subject,
thereby prognosing the subject with chronic lymphocytic leukemia (CLL).

2. A method of determining the response of a subject with chronic lymphocytic leukemia (CLL) to treatment with fludarabine, comprising,
(a) obtaining a biological sample from the subject;
(b) determining the sequence of a portion of the SF3B1 gene in the sample, wherein the portion of the SF3B1 gene comprises a sequence that encodes for a HEAT3, HEAT4 or HEAT5 domain;
(c) analyzing the sequence of the SF3B1 gene for a mutation,
wherein the presence of the mutation in the sequence of the SF3B1 gene indicates that the subject is resistant or refractory to fludarabine,
thereby determining the response of the subject with chronic lymphocytic leukemia (CLL) to treatment with fludarabine.

3. A method of prognosing a subject with chronic lymphocytic leukemia (CLL), the method comprising,
(a) obtaining a biological sample from the subject;
(b) determining the sequence of a portion of the SF3B1 polypeptide in the sample, wherein the portion of the SF3B1 polypeptide comprises the sequence of a HEAT3, HEAT4 or HEAT5 domain;
(c) analyzing the sequence of the SF3B1 polypeptide for a mutation,
wherein the presence of the mutation in the sequence of the SF3B1 polypeptide predicts a decreased survival of the subject,
thereby prognosing the subject with chronic lymphocytic leukemia (CLL); or
a method of determining the response of a subject with chronic lymphocytic leukemia (CLL) to treatment with fludarabine, the method comprising
(a) obtaining a biological sample from the subject;
(b) determining the sequence of a portion of the SF3B1 polypeptide in the sample, wherein the portion of the SF3B1 polypeptide comprises the sequence of a HEAT3, HEAT4 or HEAT5 domain;
(c) analyzing the sequence of the SF3B1 polypeptide for a mutation,
wherein the presence of the mutation in the sequence of the SF3B1 polypeptide indicates that the subject is resistant or refractory to fludarabine,
thereby determining the response of the subject with chronic lymphocytic leukemia (CLL) to treatment with fludarabine.

4. The method of claim 1, wherein the decreased survival comprises treatment-free survival or overall survival.

5. The method of claim 2, wherein treatment with fludarabine is discontinued.

6. The method of claim 2, wherein treatment with fludarabine is replaced by treatment with chlorambucil, cyclophosphamide, rituximab, alemtuzumab, bendamustine, or a combination thereof.

7. The method of claim 2, wherein treatment with fludarabine is replaced by treatment with alemtuzumab.

8. The method of claim 2, wherein the subject is treated with chlorambucil, cyclophosphamide, rituximab, alemtuzumab, bendamustine, or a combination thereof.

9. The method of claim 1, wherein the analyzing step further comprises polymerase chain reaction (PCR), DNA sequencing, or a combination thereof.

10. The method of claim 1, wherein the subject has been diagnosed with chronic lymphocytic leukemia (CLL).

11. The method of claim 1, wherein the biological sample comprises an isolated and purified genomic DNA, cDNA, or RNA molecule.

12. The method of claim 3, wherein the biological sample comprises an isolated and purified polypeptide molecule.

13. The method of claim 3, the portion of the SF3B1 polypeptide comprising the sequence of a HEAT3, HEAT4 or HEAT5 domain is selected from the group consisting of SEQ ID NO: 1, 2, 3, 4, 5, 6, 7, 8, 9, 10, 11, 12, 13, 14, 15, 16, and 19.

14. The method of claim 3, the portion of the SF3B1 polypeptide comprising the sequence of a HEAT3, HEAT4 or HEAT5 domain is SEQ ID NO: 1 or 19.

15. The method of claim 1, wherein the mutation is a missense mutation or an in-frame deletion.

16. The method of claim 1, wherein the mutation is a substitution of a Guanine (G) for an Adenine (A) at nucleotide base position 2044 or 2146 of SEQ ID NO: 17; or
the mutation is a substitution of a Thymine (T) for a Guanine (G) at nucleotide base position 2046 of SEQ ID NO: 17; or
the mutation is a substitution of an Aldenine (A) for a Guanine (G) at nucleotide base position 2267 of SEQ ID NO: 17; or
the mutation is a substitution of a Thymine (T) for an Adenine (A) at nucleotide base position 1938 of SEQ ID NO: 17.

17. The method of claim 2, wherein the mutation is a substitution of an Adenine (A) for a Cytosine (C) at nucleotide base position 2034 of SEQ ID NO: 17; or
the mutation is a substitution of a Guanine (G) for a Cytosine (C) at nucleotide base position 2032 of SEQ ID NO: 17; or
the mutation is a substitution of a Guanine (G) for an Adenine (A) at nucleotide base position 2044 of SEQ ID NO: 17; or
the mutation is a substitution of a Guanine (G) for an Adenine (A) at nucleotide base position 2146 of SEQ ID NO: 17; or
the mutation is a deletion of the nucleotide sequence CAGAAA corresponding to nucleotide base positions 2143 to 2148 of SEQ ID NO: 17; or
the mutation is a substitution of a Guanine (G) for a Cytosine (C) at nucleotide base position 2056 of SEQ ID NO: 17.

18. The method of claim 3, wherein the method prognoses a subject with CLL and wherein
the mutation results in a substitution of a Glutamic Acid (Glu or E) residue for a Lysine (Lys or K) residue at codon 666 or 700 of SEQ ID NO: 19; or
the mutation results in a substitution of an Asparagine (Asn or N) residue for a Lysine (Lys or K) residue at codon 666 of SEQ ID NO: 19; or
the mutation results in a substitution of a Glutamic Acid (Glu or E) residue for a Glycine (Gly or G) residue at codon 740 of SEQ ID NO: 19.

19. The method of claim 4, wherein the method determines the response of a subject with CLL to treatment with fludarabine and wherein
the mutation results in a substitution of a Serine (Ser or S) residue for an Arginine (Arg or R) residue at codon 630 of SEQ ID NO: 19; or
the mutation results in a substitution of a Glutamine (Gln or Q) residue for a Histidine (His or H) residue at codon 662 of SEQ ID NO: 19; or
the mutation results in a substitution of an Aspartic Acid (Asp or D) residue for a Histidine (His or H) residue at codon 662 of SEQ ID NO: 19; or
the mutation results in a substitution of a Glutamic Acid (Glu or E) residue for a Lysine (Lys or K) residue at codon 666 or 700 of SEQ ID NO: 19; or
the mutation results in a deletion of a Glutamine (Gln or Q) residue at codon 699 and a Lysine (Lys or K) residue at codon 700 of SEQ ID NO: 19; or
the mutation results in a substitution of a Glutamic Acid (Glu or E) residue for a Glutamine (Gln or Q) residue at codon 670 of SEQ ID NO: 19.


SEQUENCE LISTING (20 sequences)

SEQ ID NO: 1 - protein, 113 aa, Homo sapiens
ARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGK

SEQ ID NO: 2 - protein, 113 aa, Pan troglodytes; identical to SEQ ID NO: 1

SEQ ID NO: 3 - protein, 113 aa, Canis familiaris; identical to SEQ ID NO: 1

SEQ ID NO: 4 - protein, 113 aa, Bos taurus; identical to SEQ ID NO: 1

SEQ ID NO: 5 - protein, 113 aa, Mus musculus; identical to SEQ ID NO: 1

SEQ ID NO: 6 - protein, 113 aa, Rattus norvegicus; identical to SEQ ID NO: 1

SEQ ID NO: 7 - protein, 113 aa, Gallus gallus; identical to SEQ ID NO: 1

SEQ ID NO: 8 - protein, 113 aa, Danio rerio; identical to SEQ ID NO: 1

SEQ ID NO: 9 - protein, 113 aa, Drosophila melanogaster
ARAFAVVASALGIPSLLPFLKAVCKSKKSWQARHTGIKIVQQIAILMGCAILPHLKALVEIIEHGLVDEQQKVRTITALAIAALAEAATPYGIESFDSVLKPLWKGIRTHRGK

SEQ ID NO: 10 - protein, 113 aa, Anopheles gambiae
ARAFAVVASALGIPSLLPFLKAVCKSKKSWQARHTGIKIVQQIAILMGCAILPHLKSLVEIIEHGLVDEQQKVRTITALALAALAEAATPYGIESFDSVLKPLWKGIRTHRGK

SEQ ID NO: 11 - protein, 113 aa, Caenorhabditis elegans
ARAFAVVASALGIPALLPFLKAVCKSKKSWQARHTGIKIVQQMAILMGCAVLPHLKALVDIVESGLDDEQQKVRTITALCLAALAEASSPYGIEAFDSVLKPLWKGIRMHRGK

SEQ ID NO: 12 - protein, 113 aa, Schizosaccharomyces pombe
ARAFSVVASALGVPALLPFLKAVCRSKKSWQARHTGVRIIQQIALLLGCSILPHLKNLVDCIGHGLEDEQQKVRIMTALSLSALAEAATPYGIEAFDSVLKPLWSGVQRHRGK

SEQ ID NO: 13 - protein, 113 aa, Magnaporthe oryzae
ARAFAVVASALGIPALLPFLQAVCRSKKSWQARHTGVKIVQQIPILMGCAVLPHLKRLVDCIGPNLNDEQTKVRTVTSLAIAALAEAANPYGIESFDDILNPLWTGARKQRGK

SEQ ID NO: 14 - protein, 113 aa, Myristica crassa
ARAFAVVASALGIPALLPFLRAVCRSKKSWQARHTGVKIVQQIPILMGCAVLPHLKQLVDCIGPNLNDEQTKVRTVTSLAIAALAEASNPYGIESFDDILNPLWTGARKQRGK

SEQ ID NO: 15 - protein, 113 aa, Arabidopsis thaliana
ARAFSVVASALGIPALLPFLKAVCQSKRSWQARHTGIKIVQQIAILIGCAVLPHLRSLVEIIEHGLSDENQKVRTITALSLAALAEAAAPYGIESFDSVLKPLWKGIRSHRGK

SEQ ID NO: 16 - protein, 113 aa, Oryza sativa
ARAFSVVASALGTPALLPFLKAVCQSKKSWQARHTGIKIVQQIAILMGCAVLPHLKSLVEIIEHGLSDENQKVRTITALSLATLAEAAAPYGIESFDTVLKPLWKGIRSHRGK

SEQ ID NO: 17 - DNA, 4314 bp, Homo sapiens
ggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaaaaaaaaaaaaaaaaaaaaaaaa

SEQ ID NO: 18 - DNA, 647 bp, Homo sapiens
ggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaaaaaaaaaaaaaaaaaaaaaaaaaaaaaa

SEQ ID NO: 19 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 20 - protein, 144 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGFYSAA